(19) United States
(12) Patent Application Publication
(10) Pub. No.: US 2024/0365971 A1
(43) Pub. Date: Nov. 7, 2024

(54) SANITIZING APPARATUS, STATION, AND METHOD OF USE

(71) Applicant: Bruce Glenn, East Rockaway, NY (US)

(72) Inventor: Bruce Glenn, East Rockaway, NY (US)

(21) Appl. No.: 18/687,020

(22) PCT Filed: Aug. 31, 2022
(86) PCT No.: PCT/US2022/042119

§ 371 Date: Feb. 27, 2024

Related U.S. Application Data
(60) Provisional application No. 63/239,583, filed on Sep. 1, 2021.

(51) Int. Cl. A46B 17/06; A61L 2/04; A61L 2/07; A61L 2/10; A61L 2/26; B08B 3/02

(52) U.S. Cl. CPC A46B 17/065; A61L 2/04; A61L 2/07; A61L 2/10; A61L 2/26; B08B 3/02; A61L 2202/11; A61L 2202/121; A61L 2202/122; A61L 2202/14; A61L 2202/16; A61L 2202/17; B08B 2203/007; B08B 2230/01

(57) ABSTRACT

A sanitation device and system. The sanitation station device may include a plurality of support compartments; a heated, pressurized steam solution washing assembly; and a germicidal light illumination. The result is a hybrid hygiene sanitation station for easily, and consistently, sanitizing personal products and external manipulation. The sanitation station may be an independent, portable device adapted to align on a bathroom surface. The sanitation may include a night light and auto speaker and may include an open door or low solution indicator.

This application claims the benefit of U.S. provisional application No. 63/239,583, filed Sep. 1, 2021, which is incorporated herein by reference in its entirety.

### BACKGROUND

### FIELD

The present disclosure relates generally to hygiene practices, and more particularly to improved personal apparatus sanitization station and devices, systems, and methods.

### SUMMARY

Applicant desires a sanitation device, system, and method without the drawbacks presented by the traditional systems and methods. Those skilled in the art having the benefit of this disclosure will recognize additional features, advantages over conventional systems, and improvements for personal hygiene practices.

In accordance with the present inventions, a sanitation station is provided for a plurality of hygiene practices, including, but not limited to, sanitization and cleansing. These inventions provide an improved hybrid device and system that is convenient, efficient, and safe for the user, particularly when used in consistently ensuring proper hygiene practices.

In one embodiment, a device comprises an oval housing having at least one concave access door and supporting a plurality of internal compartments adapted to receive a plurality of personal hygiene products; and an adjustable sanitizing assembly positioned within the housing, wherein the adjustable sanitizing assembly includes a heated, pressurized solution washing assembly in fluid communication with the internal compartments; and a germicidal UV light illumination in optical communication with the support compartments, and wherein the sanitizing assembly to sanitize at least one personal hygiene product being accessible about the concave access door.

In one embodiment, a device comprises an oval housing having at least one concave access door and supporting a plurality of internal compartments adapted to receive a plurality of personal hygiene products; an adjustable sanitizing assembly positioned within the housing, wherein the adjustable sanitizing assembly comprises a heated, pressurized solution washing assembly in fluid communication with the internal compartments; and a germicidal UV light illumination in optical communication with the support compartments; and a removable solutionpik to deliver a solution stream, and wherein the sanitizing assembly to sanitize at least one personal hygiene product being accessible about the concave access door.

In one embodiment, a hygiene device comprises a plurality of support compartments to receive a personal hygiene product; a heated, pressurized solution washing assembly in fluid communication with the support compartments; and a germicidal light illumination in optical communication with the support compartments.

In certain examples, the device includes a solutionpik to deliver a solution stream. The solutionpik may include a tube in fluid communication with a solution reservoir. The plurality of support compartments may include at least one toothbrush aperture adapted to temporarily secure a neck of a toothbrush in an operating position. The toothbrush aperture may include a knob and spring-loaded system adapted to provide a pressure against the neck of the toothbrush. The device may include a plurality of toothbrush apertures. The plurality of toothbrush apertures may be spatially aligned adjacent one another. The plurality of support compartments may include at least one razor aperture. The device may include a plurality of razor apertures.

In certain examples, the heated, pressurized solution washing assembly is adapted to provide a pressurized fluid spray of greater than about one hundred pounds per square inch, for instance about an individual spray nozzle, distributed about a plurality of spray nozzles, while those skilled in the art will recognize any combination or individual amount of pressurized fluid, spray, and the like. The device may include a switch to toggle between a desired solution pressure. The heated, pressurized solution washing assembly may include a tank configured to store washing solution supplied to the support compartments. The heated, pressurized solution washing assembly may include a pump adapted to supply a washing solution stored in the tank to a spray nozzle. The heated, pressurized solution washing assembly may include at least one pressurized output port adapted to direct a pressurized fan of solution at the personal hygiene product. The heated, pressurized solution washing assembly may include a solution heating device coupled to a solution pump and adapted to heat the solution to a desired effluent temperature. The desired effluent temperature may include about one hundred and forty degrees Fahrenheit.

In certain examples, the solution comprises water. While other examples include any liquid, solution, combination of active ingredients, and the like.

In certain examples, the heated, pressurized solution washing assembly may include a plurality of spray nozzles. The heated, pressurized solution washing assembly may include a port in fluid communication with a manifold adapted to selectively direct solution to associated nozzles. The heated, pressurized solution washing assembly may include a nozzle adapted to project a pressurized steam. The pressurized steam may be adapted to wash at least one chosen from a debris on a toothbrush and a debris on a razor. The device may include a Y-split tubing having respective distal nozzles.

In certain examples, the germicidal light illumination directs a sanitizing illumination at the support compartments. The germicidal light illumination may include at least one ultraviolet (UV) light source adapted to project a UV light for at least one of disinfection, sanitization, and sterilization of the personal hygiene product. The germicidal light illumination may be adapted to provide a dosage of UV light emitted by the UV light source to ensure the personal hygiene product is at least one of disinfected, sanitized, and sterilized. The UV light source may be adapted to emit UV light having a wavelength from about 50 nanometers to about 500 nanometers. The germicidal light illumination may include a UV light source of at least one of a UV light emitting diode (LED), UV LED array, fluorescent bulb, a laser, and a combination thereof.

In certain examples, the device includes a housing. The housing may include an oval shape. The device may include an activation assembly. The device may include a funnel, for instance adapted to distribute a solution to the heated, pressurized solution washing assembly. The hygiene device may be an independent, portable device, while other examples include a rigid, permanent fixture. The plurality of support compartments may be independent. The independent support compartments may include at least one toothbrush support compartment being independent of a razor support compartment, and whereby the independent support compartments adapted to eliminate cross contamination of the toothbrush support compartment about the razor support compartment.

In one embodiment, a sanitation station comprises a housing having at least one access door; a sanitizing assembly housed within the housing and adapted to sanitize at least one personal hygiene product being accessible about the access door; and an activation assembly integrated about the housing and adapted to activate the sanitizing assembly.

In certain examples, the housing includes an upper switch gate, wherein the upper switch gate comprises: a first portion release mechanism adapted to release the access door; and a second portion aperture adapted to expose a solution fill inlet. The assembly may be adapted to provide a sanitization to ensure the personal hygiene product is at least one of disinfected, sanitized, and sterilized according to a corresponding output indication. The housing may include an oval shape. The access door may have a concave elongated surface extending along an upper portion of the oval housing.

In certain examples, the housing may include a non-oval shape.

In certain examples, the housing may include a night light. The assembly may include a motion sensor in electrical communication with the night light. The assembly may include a plurality of color illuminations. The assembly may include a plurality of adjustable brightness levels. The assembly may include a slide cover. The slide cover may include a spring-loaded slide cover adapted to release in an opening position and apply pressure in a closed position. The housing may include a discharge tube. The discharge tube may include a plurality of venting holes adapted to evaporate moisture.

In certain examples, activation assembly may include a user control interface. The user control interface may include a control adapted to activate a heated, pressurized solution washing assembly. The user control interface may be adapted to activate the heated, pressurized solution washing assembly in an individual chamber. The user control interface may include a control adapted to activate a germicidal light illumination. The user control interface may be adapted to activate germicidal light illumination in an individual chamber.

In certain examples, the housing comprises a low solution tank indicator. The housing may include an open door indicator. The housing may include at least one external support slot. For instance, the assembly may include a toothpaste slot. The housing may include an audio speaker adapted to provide at least one sound effect.

In certain examples, the sanitizing assembly may be adapted to provide a pressurized fluid sanitization to ensure the personal hygiene product is at least one of disinfected, sanitized, and sterilized according to a corresponding output indication. The sanitizing assembly may include a fluid sanitizing system. The sanitizing assembly may include at least one pressurized output port adapted to direct a pressurized stream of solution at the personal hygiene product. The sanitizing assembly may include a germicidal light illumination adapted to direct a sanitizing illumination at the personal hygiene product. The germicidal light illumination may include at least one UV light source adapted to project a UV light for at least one of disinfection, sanitization, and sterilization of the personal hygiene product. The sanitation station may be an independent, portable device adapted to align on a surface.

The above summary was intended to summarize certain embodiments of the present disclosure. Embodiments will be set forth in more detail in the figures and description of embodiments below. It will be apparent, however, that the description of embodiments is not intended to limit the present inventions, the scope of which should be properly determined by the appended claims.

### BRIEF DESCRIPTION OF THE DRAWINGS

Embodiments of the disclosure will be better understood by a reading of the Description of Embodiments along with a review of the drawings, in which:

FIG. 1 is a top perspective view of a sanitizing device according to one embodiment of the disclosure;

FIG. 1A is a top perspective view of a sanitizing device according to one embodiment of the disclosure;

FIG. 2 is a side plan view of the exemplary apparatus shown in FIG. 1;

FIG. 2A is a side plan view of the exemplary apparatus shown in FIG. 1A;

FIG. 3 is a front plan view of the exemplary apparatus shown in FIG. 1;

FIG. 3A is a front plan view of the exemplary apparatus shown in FIG. 1A

FIG. 4 is a side plan view of the exemplary apparatus shown in FIG. 1;

FIG. 4A is a side plan view of the exemplary apparatus shown in FIG. 1A;

FIG. 5 is a top plan view of the exemplary apparatus shown in FIG. 1, with elements removed for clarity;

FIG. 5A is a top plan view of the exemplary apparatus shown in FIG. 1A

FIG. 5B is a top plan view of the exemplary apparatus shown in FIG. 1;

FIG. 5C is a rear plan view of the exemplary apparatus shown in FIG. 1;

FIG. 5D is a rear plan view of the exemplary apparatus shown in FIG. 1;

FIG. 5E is an exploded front view of the exemplary apparatus shown in FIG. 1, with elements removed for clarity;

FIG. 5F is a cross sectional view of the exemplary apparatus shown in FIG. 1, with elements removed for clarity;

FIG. 5G is a cross sectional view of the exemplary apparatus shown in FIG. 1, with elements removed for clarity;

FIG. 5H is a cross sectional view of the exemplary apparatus shown in FIG. 1, with elements removed for clarity;

FIG. 6 is a perspective view of one embodiment of a sanitation station with the exemplary apparatus shown in FIG. 1;

FIG. 7 is a front view of the embodiment shown in FIG. 6;

FIG. 8 is a rear view of the embodiment shown in FIG. 6;

FIG. 9 is a side view of the embodiment shown in FIG. 6;

FIG. 10 is a side view of the embodiment shown in FIG. 6, with elements removed for clarity;

FIG. 11 is a side view of the embodiment shown in FIG. 6 in an open position, with elements removed for clarity;

FIG. 12 is a front perspective view of the embodiment shown in FIG. 6 in an open operating position, with elements removed for clarity;

FIG. 13 is a top view of the embodiment shown in FIG. 6; and

FIG. 14 is a side perspective view of the embodiment shown in FIG. 6 in an open position, with elements removed for clarity.

### DETAILED DESCRIPTION

In the following description, like reference characters designate like or corresponding parts throughout the several views. Also in the following description, it is to be understood that such terms as “forward,” “rearward,” “left,” “right,” “upwardly,” “downwardly,” and the like are words of convenience and are not to be construed as limiting terms.

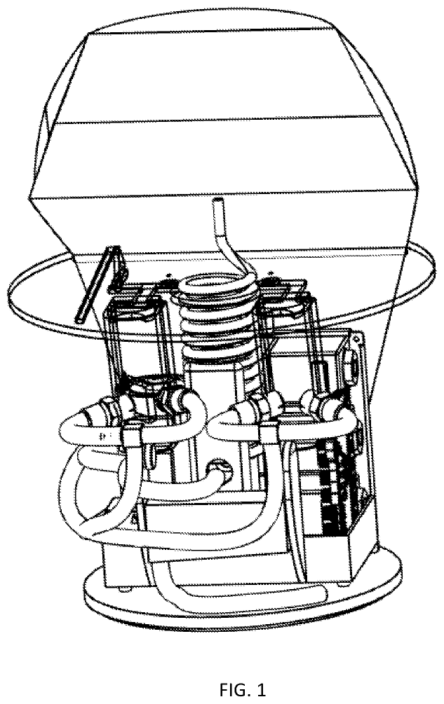

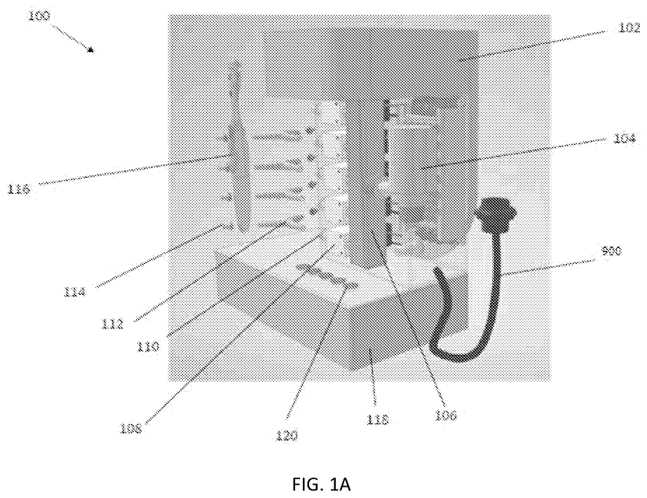

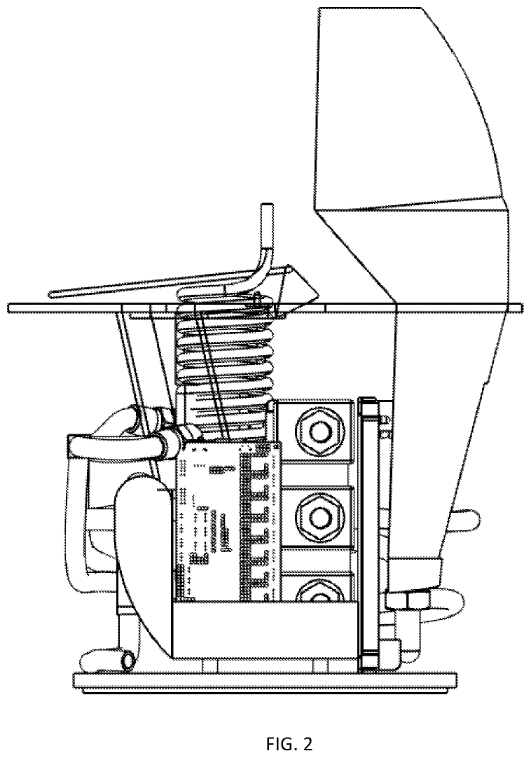

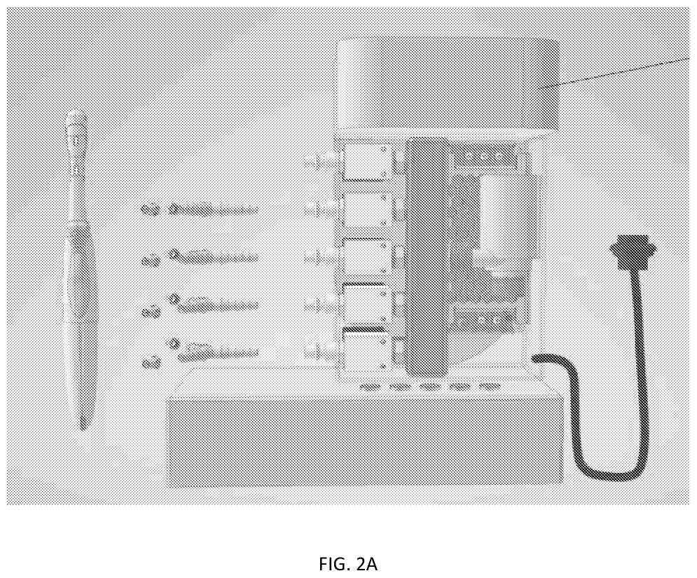

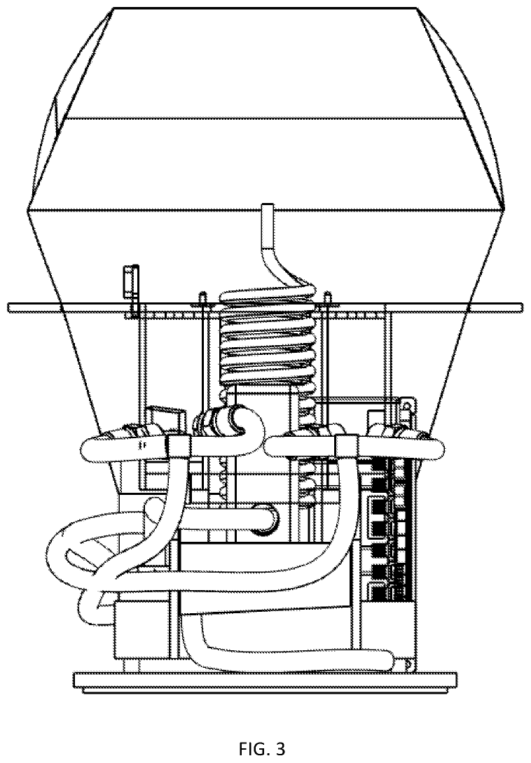

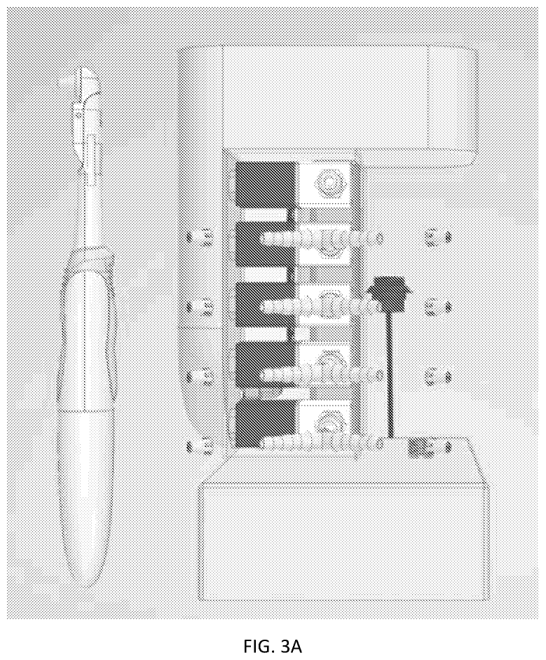

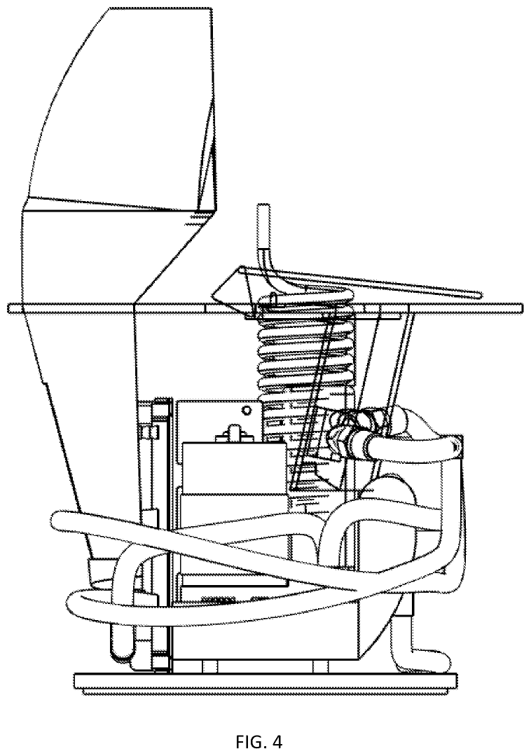

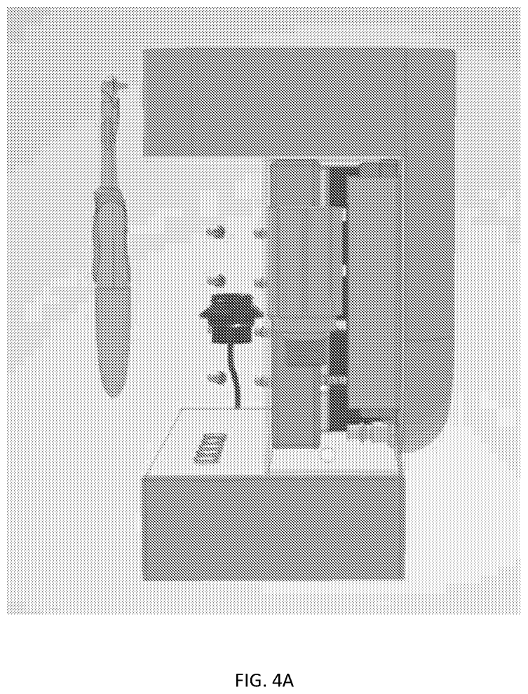

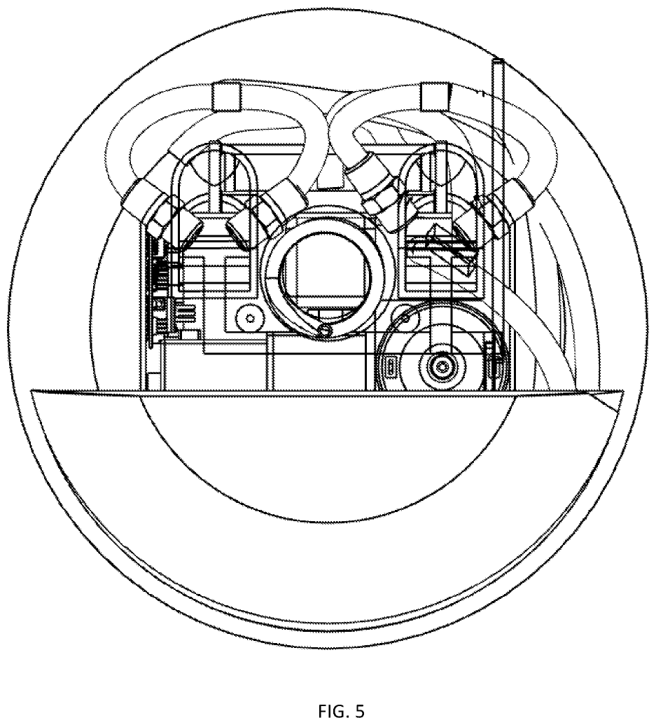

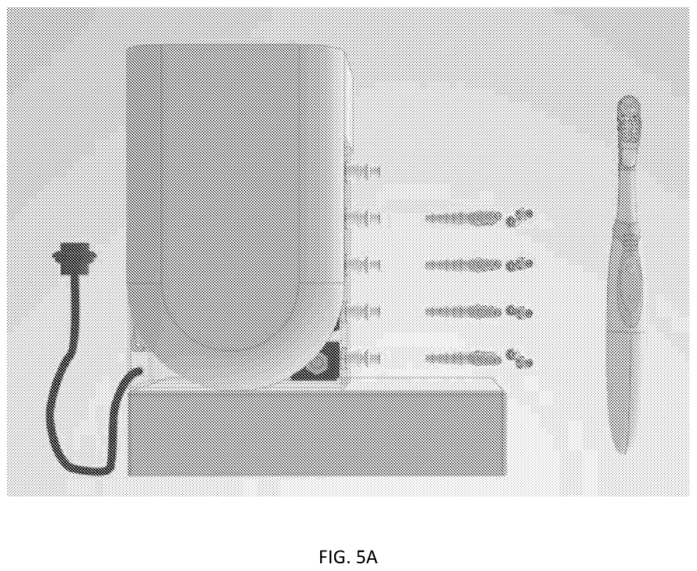

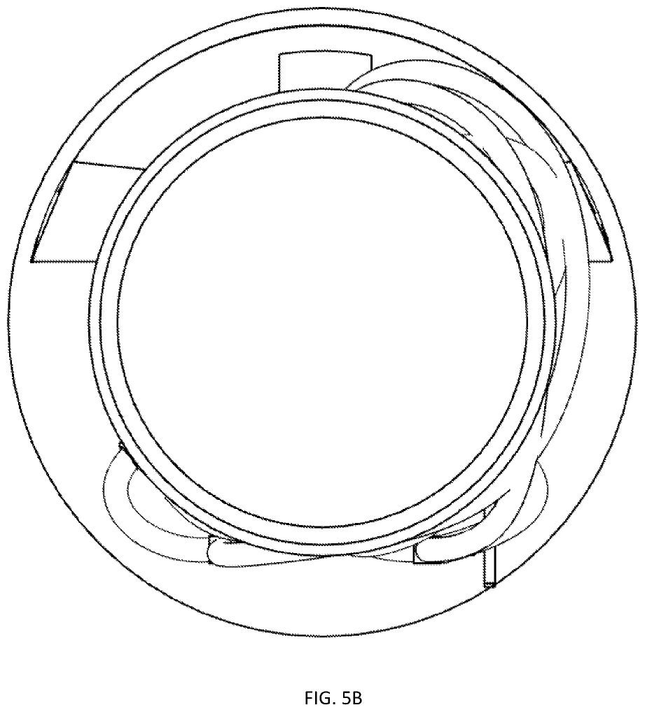

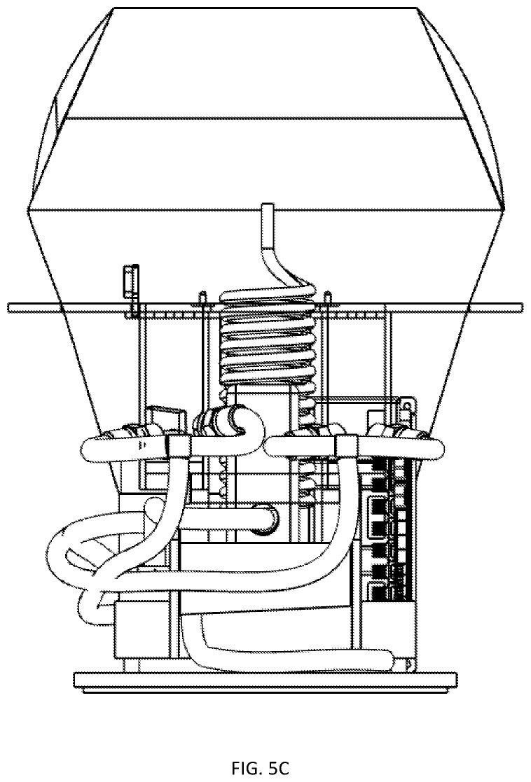

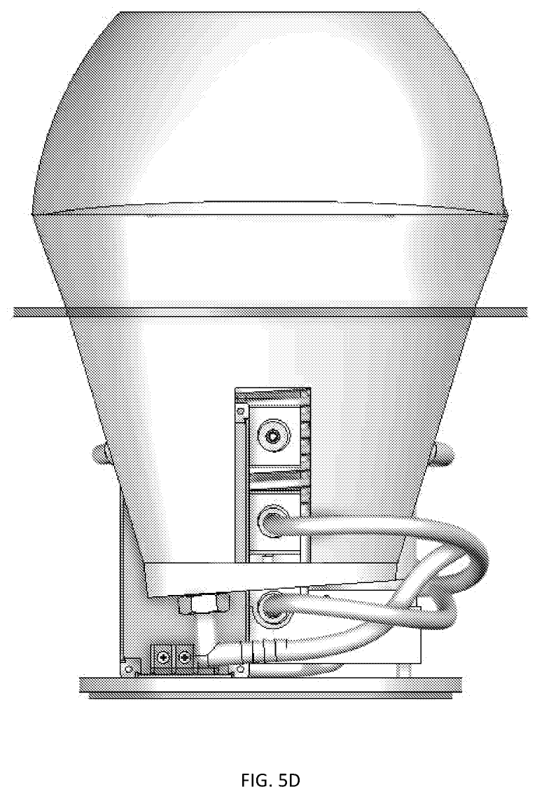

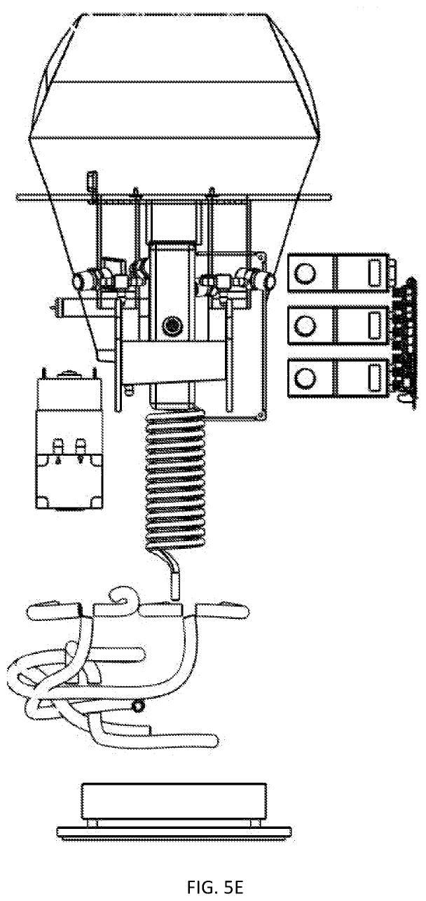

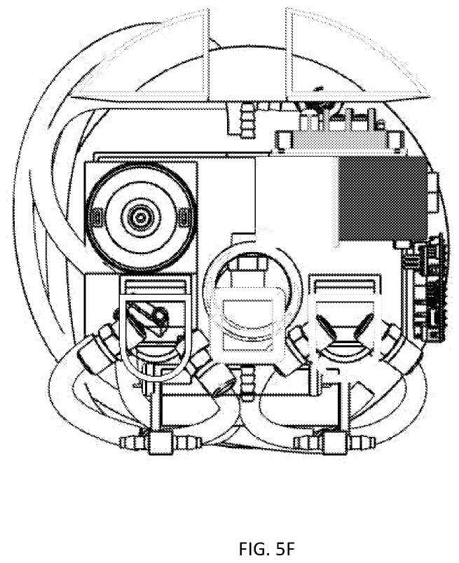

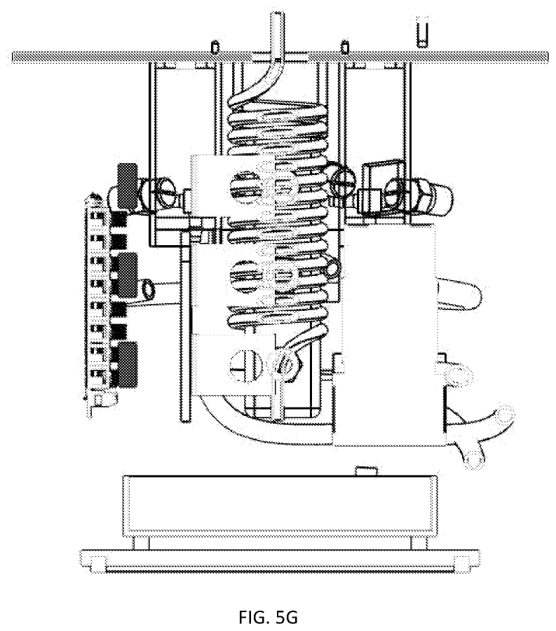

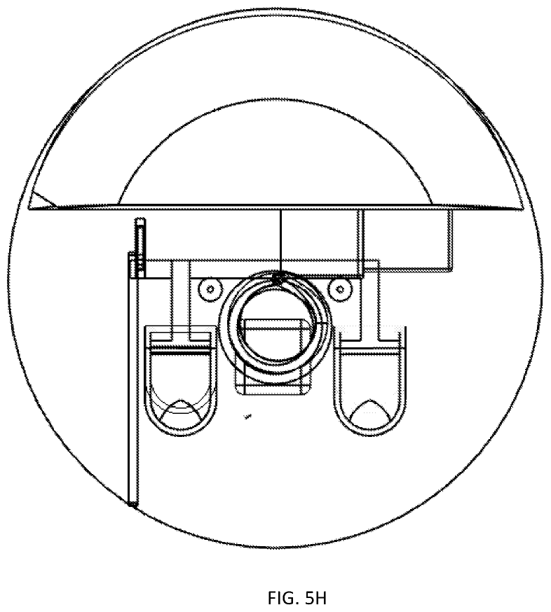

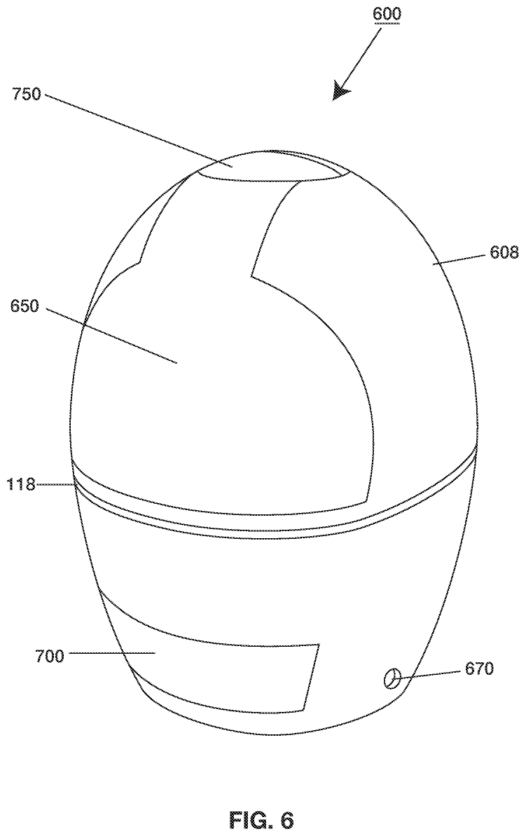

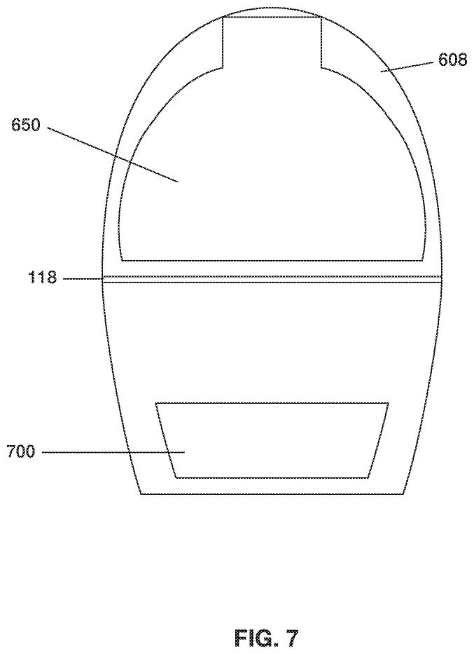

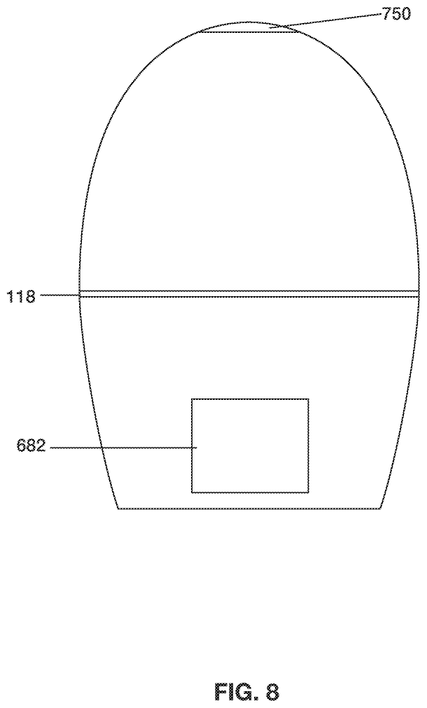

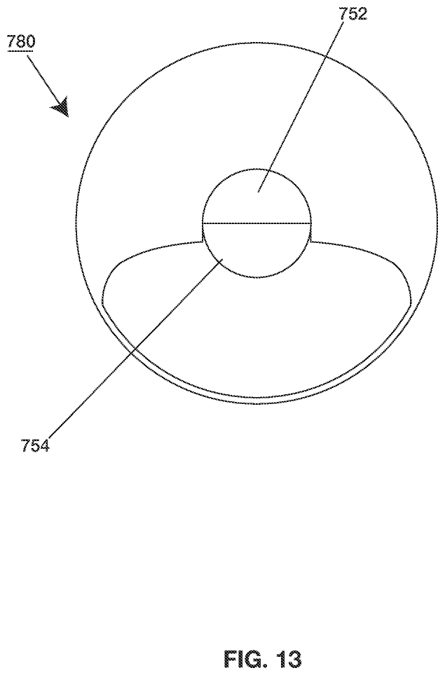

Referring now to the drawings in general and FIGS. 1 and 6 in particular, it will be understood that the illustrations are for the purpose of describing embodiments of the disclosure and are not intended to limit the disclosure or any inventions thereto. As seen in FIGS. 1-14, the universal sanitization station device 100 provides improved hygiene and sanitation. As shown in the figures, the device and assemblies may generally include a water tank 102, water pump 104, spray nozzles 114 to direct pressurized solution, steam, and the like to personal products in an operating position, and housing outer casing 608. Those skilled in the art having the benefit of this disclosure will recognize additional sizes, orientations, material, and arrangement of elements; including, but not limited to supporting a variety of differing personal products, including toothbrushes, razors, and the like.

In one embodiment, a sanitation station 100 comprises a housing 608 with an interior chamber 602 housing any of the elements and examples shown and described herein. As illustrated, the sanitation station 100 may have at least one access door 650; a sanitizing assembly housed within the housing 608 to sanitize at least one personal hygiene product shown and described herein and being accessible about the access door 650; and an activation assembly integrated about the housing 608 to activate any alert, sanitizing assembly, or system shown and described herein.

As shown, device 100 may include an oval housing 608 having at least one concave access door 650 and supporting a plurality of internal compartments to receive any personal hygiene product shown and described herein. The device 100 may include an adjustable sanitizing assembly positioned within the housing, for instance an adjustable sanitizing assembly comprising a heated, pressurized solution washing assembly in fluid communication with the internal compartments; and a germicidal UV light illumination in optical communication with the support compartments.

As illustrated, the hygiene device 100 comprises a plurality of support compartments to receive any personal hygiene product shown and described herein; including, but not limited to, toothbrushes and razors. The hygiene device 100 may include a heated, pressurized solution washing assembly in fluid communication with the support compartments for a variety of time spans, including, but not limited to, seconds and minutes of pressurized solution washing for any particular application shown and described herein; and a germicidal light illumination in optical communication with the support compartments for a variety of time spans, including, but not limited to, seconds and minutes of germicidal light illumination for any particular application shown and described herein. Those skilled in the art having the benefit of this disclosure will recognize additional scaling, elemental orientation, and arrangements within the spirit of this disclosure.

As shown, device 100 may have an oval housing 608 having at least one concave access door 605 and supporting any of the internal compartments shown and described herein to receive any personal hygiene product. The hygiene device 100 may include an adjustable sanitizing assembly that is generally positioned within the housing 608. In certain examples, the adjustable sanitizing assembly includes a heated, pressurized solution washing assembly in fluid communication with the internal compartments; and a germicidal UV light illumination in optical communication with the support compartments. Further, as shown and described herein in certain examples the device 100 includes a removable solutionpik to deliver a solution stream.

As shown in FIG. 1, the toothbrush sanitizing apparatus 100 will generally include a water tank 102 that is fluidly connected or coupled with a water pump 104. The position of the particular components of the apparatus 100 shown in FIG. 1 are merely exemplary. In alternate embodiments, the components of the apparatus 100 can be arranged and connected in any suitable manner.

As illustrated in the Figures, the water pump 104 is fluidly connected to two or more spray nozzles 114. The pump 104 is configured to deliver the water from the tank 102 to the nozzles 114 at a desired pressure. In one embodiment, the pump 104 is configured to deliver over 100 PSI to the individual spray nozzles 112. Although 100 PSI is generally described herein as a desired water pressure, the aspects of the disclosed embodiments are not so limited. In alternate embodiments, the pump 104 can be configured to deliver the water at any desired or suitable pressure, other than including 100 PSI. For example, more or less pressure may be desired based upon the application or type of toothbrush or razor blade. More pressure may be desired or used on a razor blade, while less pressure may be desired with the bristles of a toothbrush. In one embodiment, the apparatus 100 might include a switch for selection of a desired water pressure, such as one pressure for a razor blade and one pressure for a toothbrush.

As shown in FIG. 1, the water pump 104 is connected to a manifold 106. One or more solenoids 108 can be used to selectively allow water from the pump 104 to be directed to the associated spray nozzles 114. Further, shown in FIG. 1, a solenoid 108 is connected to a port 110. In the example of FIG. 1, the water from the pump 104 is divided into five individual ports 110. The port 110 is fluidly connected to tubing that includes a Y-splitter 112. A single line or port 110 is split into two. Although the description herein shows the port 110 being divided into two separate lines, the aspects of the disclosed embodiments are not so limited. In alternate embodiments, a port 110 can be divided into any suitable number of lines, other than including two. For example, a port 110 can be maintained as a single line. Alternatively, a port 110 can be divided into more than two lines, such as three or more. In this manner, each line or port 110 can be configured to deliver one or more lines of pressurized and/or heated water to a nozzle or jet that is disposed in a chamber, as is generally described herein.

As illustrated in the various figures, and FIG. 1 in particular, the respective ends of the Y-splitter 112 may include respective nozzles 114. Although the example of FIG. 1 shows an arrangement of five ports 110 and nozzles 114, in alternate embodiments, the apparatus 100 can include any suitable or desired number of ports 110 and nozzles 114, other than including five. For example, the apparatus 110 may include only one port 110 and nozzles 114. Where there is more than one port 110 and associated nozzles 114, the apparatus 100 can be configured to clean and disinfect more than one toothbrush head at the same time.

In the example of FIG. 1, there are four ports 110 for toothbrush cleaning. The fifth port can be used for a hand-held wand that can be used to deliver a pressurized spray or stream(s) of water in a portable manner. Although the tubing 112 is illustrated as a rigid tubing, the aspects of the disclosed embodiments are not so limited. In alternate embodiments, any suitable tubing can be used to fluidly couple the output of the water pump 104 to a nozzle 114.

In one embodiment, a spray nozzle 114 will be configured to deliver one or more pressurized stream or spray of water. The pressure of the water, and the associated nozzle or jet 114, will be designed to effectively clean the bristles of the head of the toothbrush that is inserted into a chamber as is generally described herein. In one embodiment, the stream or spray from a nozzle or jet 114 will be in the form of a fan, rather than a direct stream. A fan will provide a wider area of contact of the pressurized stream(s) with the bristles of the toothbrush.

In one embodiment, a nozzle 114 can be in the form of a pressure jet. A pressure jet can have multiple water jet nozzles or sprays (holes) from each head. A pressure jet can also have the water sprayed in a fan like manner from each jet nozzle. In alternate embodiments, the apparatus 100 can include any suitable type, form and number of jets or nozzles that are configured to direct pressurized stream(s) of water at the head of the toothbrush to dislodge and remove particles and otherwise clean the bristles.

In one embodiment, a nozzle 114 can be configured to rotate, or provide a rotating stream or streams of water. In this manner, the pressurized stream(s) of water will contact more surfaces and areas of the head of the toothbrush.

In one embodiment, the apparatus 100 can include a base 118. One or more indicator lights 120 and/or switches can be disposed on the base 118. The indicator lights 120 can be configured to indicate an operational status of the apparatus 100. Although the illustration in FIG. 1 shows that the one or more indicators 120 are on the base 118, the aspects of the disclosed embodiments are not so limited. In alternate embodiments, the one or more indicators 120 can be at any suitable location on the apparatus 100. For example, in one embodiment, the indicator 120 can be disposed on a surface of a corresponding chamber.

In one embodiment, the apparatus 100 can include one or more switches. The one or more switches can be used to activate cleaning of a particular toothbrush head when disposed in a corresponding chamber of the apparatus 100. Although the aspects of the disclosed embodiments are described herein with respect to the indicators and switches being on the base 118, the aspects of the disclosed embodiments are not so limited. In alternate embodiments, the indicators and switches can be located at any suitable location on the apparatus 100. This can include for example, on a surface of a respective chamber.

In one embodiment, the apparatus 100 can include a heater that is configured to heat the water. The water can be heated to a temperature that is suitable to kill bacteria and promote disinfection of the head of the toothbrush and blade(s) of the razor. In one embodiment, the water can be heated to a temperature of at least 140 degrees. In alternate embodiments, the water can be heated to any suitable temperature.

In certain embodiments, the disclosed embodiments generally relate to devices for sanitizing toothbrushes. In particular, the aspects of the disclosed embodiments are directed to a toothbrush sanitizing apparatus that is configured to use a pressurized stream(s) of water to clean and sanitize the bristles of one or more toothbrushes.

In certain embodiments, the toothbrush sanitizing apparatus will include a pressure washer device. The pressure washer device will have at least one output port configured to direct one or more pressurized streams of water at or onto the head of a toothbrush. In one embodiment, the pressure washer device can be configured to deliver over 100 PSI to individual spray nozzles of the apparatus 100.

In one embodiment, the apparatus 100 can include a water heating device. The water heating device can be fluidly coupled to the pressure washer device and configured to heat the water that is delivered as the one or more streams of pressurized to a desired temperature. In one embodiment, the water heating device can be configured to heat the water to at least 140 degrees Fahrenheit in order to effectively kill bacteria and other germs; however those skilled in the art having the benefit of this disclosure will recognize alternate embodiments, the water can be heated to any suitable temperature other than including 140 degrees.

In one embodiment, the apparatus 100 can include a germicidal lighting device. The germicidal lighting device can be configured to direct ultraviolet C (UV-C) light at the head of the toothbrush. In this manner, the bristles of the head of the toothbrush are exposed to the UV-C light. Those skilled in the art having the benefit of this disclosure will recognize additional germicidal lighting scaling, elemental orientation, and arrangements within the spirit of this disclosure.

In one embodiment, the apparatus 100 will include separate chambers or compartments for one or more toothbrushes and/or toothbrush heads. The chambers will allow a user to insert at least the head portion of the toothbrush-the bristle end-into a chamber for cleaning. In one embodiment, a portion of the handle may remain exposed. In one embodiment, a chamber may include a plate member and spring arrangement that is configured to retain the head portion of the toothbrush in the chamber. In one embodiment, the chamber can include one or more guide members that are configured to guide the head portion of the toothbrush into a predetermined position within the chamber. The predetermined position is generally configured to dispose the head portion of the toothbrush in a certain position within the chamber, relative to the spray nozzles or jets that are disposed within the chamber, for cleaning the head portion of the toothbrush with the pressurized of water.

In one embodiment, at least two spray nozzles will be disposed in the separate chambers. The spray nozzles, also referred to as jets, are configured to deliver one or more pressurized streams of water inside the chamber.

In one embodiment, there will be one or more individual spray nozzles disposed within the chamber. The pressurized stream(s) of water from the nozzles will be directed to surfaces on the sides of a toothbrush head. In one embodiment, one or more germicidal light(s) will be disposed in the individual chambers in order to direct the UV-C light onto the head of a toothbrush. Any suitable type of UV-C lighting device can be incorporated into a chamber.

In one embodiment, the apparatus 100 can include a chamber or compartment that is configured to house or hold a razor. The razor holding compartment will also include one or more spray nozzles. A pressurized spray, or sprays, of water will be directed onto the blade(s) of the razor in order to remove hairs and other debris that might otherwise accumulate. In one embodiment, the germicidal light(s) will be disposed in the razor holding chamber in order to direct the UV-C light onto the blades of a razor.

In one embodiment, a chamber of the apparatus 100 will include one or more drainage ports or holes. The drainage ports are configured to direct the contaminated water from the toothbrush head or razor out of the chamber. The drainage ports will allow the chamber to be emptied of water and debris that will collect during a cleaning process. In one embodiment, a surface of the chamber can be sloped or angled to allow the water and debris to more easily drain to and from the drain port(s).

In one embodiment, the apparatus 100 includes a tank. The tanks is configured to collect water and debris that drains from the one or more chambers of the apparatus. In one embodiment, the drain port(s) or hole(s) of the chamber are fluidly coupled to the tank. In one embodiment, the tank is disposed underneath the one or more chambers. The tank can have any suitable size or shape. In one embodiment, the tank is fluidly coupled to a drain tube. In one embodiment, one or more surfaces of the tank are sloped or angled. The slope or angle will enable the tank to drain more readily into the drain tube or other drain port that may be used to empty the tank when desired.

As introduced in FIG. 6, sanitation station 600 includes a water tank disposed supported in connection with a base 118. As later shown, toothbrush 10 is disposed in a chamber or compartment 602. The chamber 602 is configured to hold the head of the toothbrush 10 while it is being pressure-washed, cleaned and disinfected as is generally described herein. In certain examples, although non-limiting, the apparatus 600 includes four chambers 602. In alternate embodiments, the apparatus 600 can include any suitable number of chambers 602, other than including four.

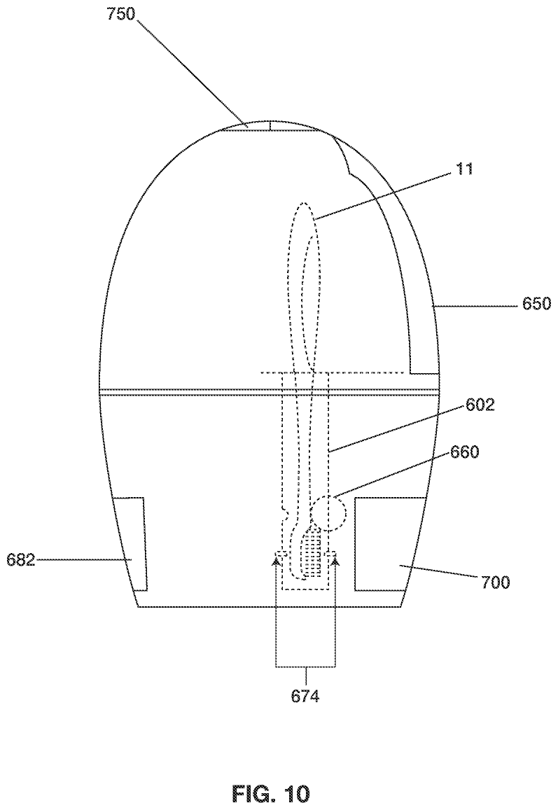

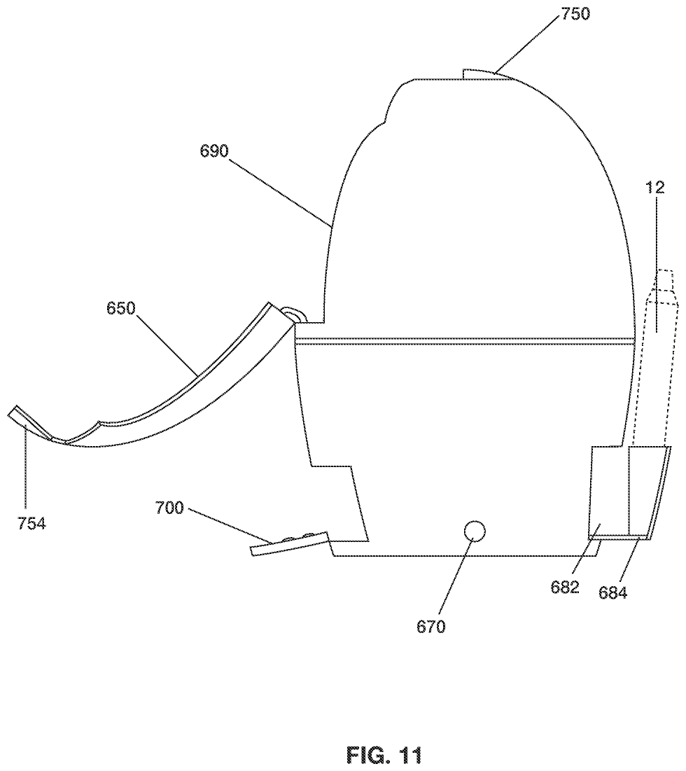

As illustrated in the embodiment in FIG. 6, a sanitation station 600 may include outer housing 608, a housing feature 670, a user interface 700, a base 118, a removeable cover 650, and switch gate 750. As introduced in FIG. 10, one embodiment includes at least one, but including a plurality, of chambers 602, including both toothbrush 10 and razor chambers, and a pressure stability system 660 for securing any of the personal products shown herein. As shown in FIG. 11, the sanitation station 600 may include an access door release 754 which is releasable from a closed position in any manner, including touch, sensor, voice activation, remote access, and the like to expose interior 690, for instance to access any of the chambers shown and described herein. The sanitation station 600 may include an external support slot 682, for instance, as illustrative purposes only, a tooth paste slot 684. In certain examples, the housing feature 670 may include any light, sensor, speaker, a combination thereof, and the like. Those skilled in the art having the benefit of this disclosure will recognize additional features.

In one embodiment, the apparatus includes a drain tube 604. The drain tube is fluidly coupled to one or more of the tank and/or chambers. The drain tube will allow the fluid and debris from the cleaning of the tooth brush head to be disposed of. In one embodiment, the drain tube is flexible and/or movable in order to allow the drain tube to be directed to a fluid disposal area, such as a sink. In one embodiment, the drain tube can be tucked out of the way when not in use.

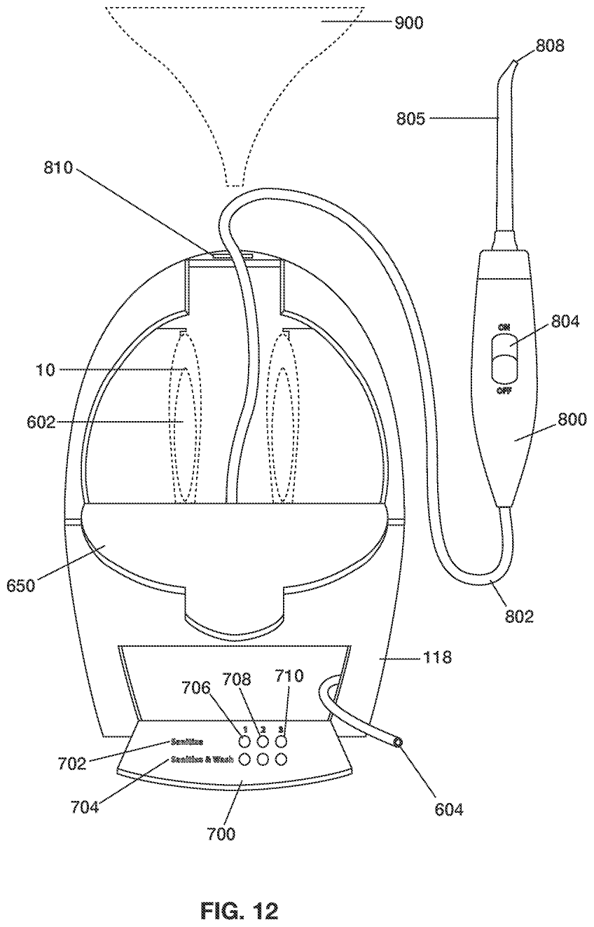

FIG. 12 illustrates one embodiment having a solutionpik 800 to support any of the pressurized solution elements and examples shown and described herein. In one embodiment the apparatus includes a wand, for instance, but not limited to, a solutionpik. The solutionpik is a handheld device and can be used to deliver one or more streams of pressurized water to a targeted area, such as the mouth. For example, the solutionpik can be used to massage the gums and remove food particles on or between the teeth. In one embodiment, the solutionpik is fluidly coupled to the apparatus via tubing. The tubing is flexible in nature and allows for the wand to be held in a person's hand and manipulated during use. As shown, the solutionpik 800 may include a connection tubing 802 in fluid communication with any solution element herein, including a pump, reservoir, and the like; a control 804, an elongated extension 806 for user application as understood by those skilled in the art having the benefit of this disclosure; and a discharge port 808.

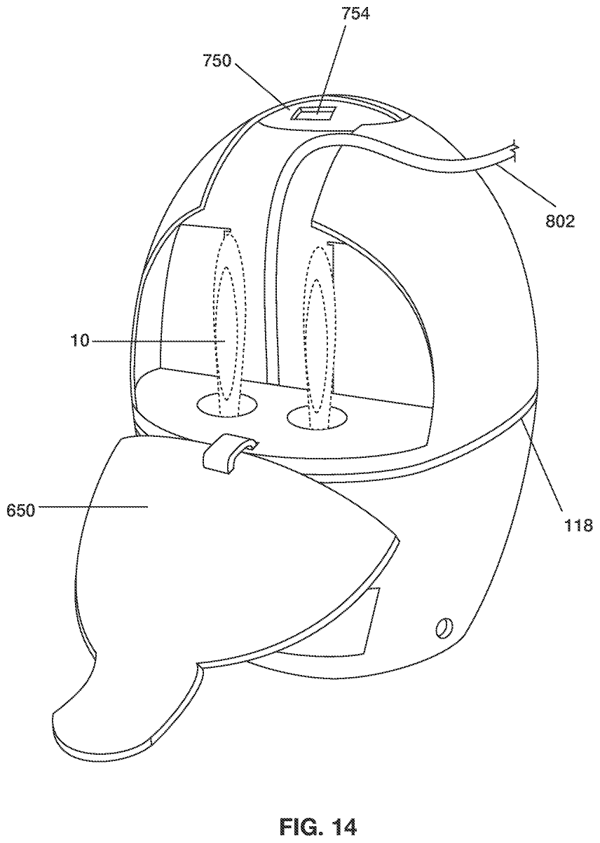

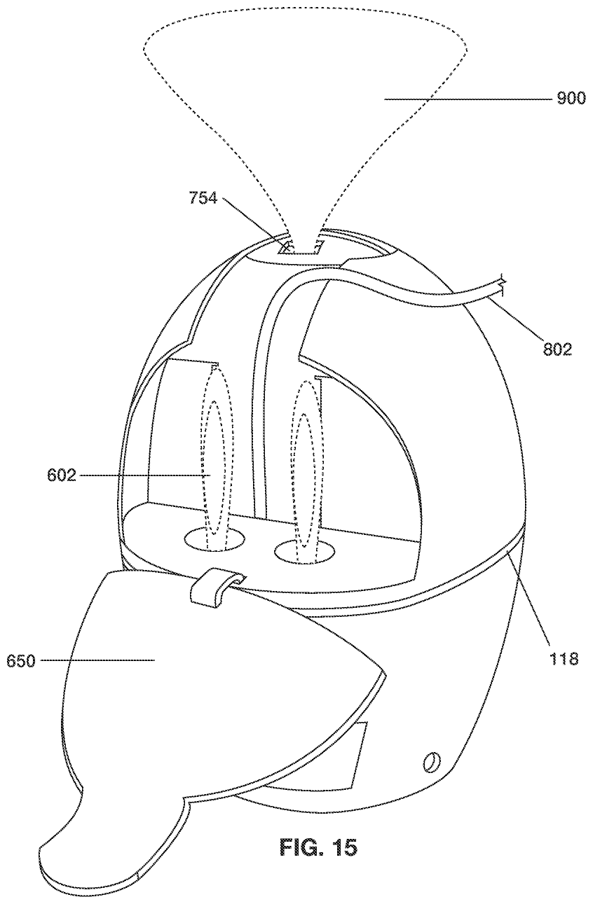

Further, as shown in FIG. 12 certain embodiments may include a funnel 900 to support any of the filling methods and elements shown and described herein, including the alignment shown about the solution fill inlet 754 in FIGS. 14-15. The embodiment in FIG. 12 further shows a user interface 700 having a plurality of operation options. In certain examples the user interface 700 may support a sanitize only 702 feature selection, i.e. activation of any of the sanitization features shown and described herein; as well as a sanitize and wash feature selection 704. Further shown, the user interface 700 may include activation of certain compartments, all compartments, and a combination thereof, for instance compartments 706, 708, 710, etc. Any of the activation assembly examples and embodiments herein may include a user interface, mechanical switch gate application, motion activation, remote activation, voice control, a combination thereof, and the like.

As described and shown herein, the chamber 602 will be fluidly connected to at least two water nozzles 114. The water nozzles or jets 114 will be disposed within an interior of a chamber 602. The water nozzles 114 are configured to direct one or more streams of pressurized water onto the head of the toothbrush 10 that is inserted into a chamber. The head of the toothbrush 10 includes the bristles.

In one embodiment, the chamber 602 is configured so that the nozzles 114 are disposed to direct pressurized water onto or at the sides of the head of the toothbrush. The nozzles 114 can also be configured to direct the pressurized stream(s) of water to dislodge debris that may collect closer to the base of the toothbrush head, where the bristles connect to the head. In one embodiment, one or more nozzles 114 can be disposed to direct pressurized water stream(s) at the front of the bristles in addition to the sides. Although the aspects of the disclosed embodiments are generally described herein with respect to two nozzles 114 in the chamber 602, the aspects of the disclosed embodiments are not so limited. In alternate embodiments the chamber 602 can include any suitable number of nozzles 114, other than including two. The nozzles 114 can be positioned at any suitable location(s) within a chamber. For example, in one embodiment it may be desirable to include more than two nozzles, based on the size of the head of the toothbrush and the desired placement of the nozzles within the chamber 602.

The nozzles 114, and associated jet stream, may be configured to effectively pressure wash and dislodge live and dead cells scraped onto the bristles of the toothbrush from a person's mouth while brushing, as well as food and other particles that may be embedded inside the bristles and the head of the toothbrush. The individual toothbrush holding chambers 602 shown in FIG. 6 are configured to eliminate any cross contamination from water, food, cells or other debris landing on an adjacent toothbrush head during the cleaning process.

In one embodiment, there are one or more germicidal lighting devices disposed in a chamber 602. The lighting devices are configured to direct UV-C light onto the bristles of the toothbrush head for disinfecting. The lighting devices are configured to be manually or automatically activated. For example, in one embodiment, a cleaning cycle can be an automated process. One or more sensors can be used to detect when a toothbrush head is disposed in a chamber 602. The one or more sensors can be configured to detect which chambers 602 have toothbrushes inserted and which do not. In this manner, pressure washing can be controlled or limited to only those chambers in which a toothbrush is properly inserted.

In particular examples of use, a cleaning cycle can start with a pressure washing of the toothbrush head. The pressure washing cycle can be any desired or suitable time period. For example, a range of one to three minutes may be suitable. Longer or shorter time ranges may also be suitable, depending upon the application. The apparatus 100 can be configured to pressure wash all inserted toothbrushes at once, individually, or as selected.

In one embodiment, once the pressure washing is complete, there can be a drying cycle. In one embodiment, one or more heaters can be used to assist the drying. In one embodiment, one or more fans may be used to air dry or otherwise assist in drying the toothbrush head(s). In one embodiment, the fan(s) and/or heater(s) can be arranged in a respective chamber. In one embodiment, the fan(s) and/or heater(s) can be arranged centrally to all of the chambers.

In one embodiment, after the pressure washing cycle is complete, there can be a UV-C light cycle. The UV-C lights will come on for a pre-determined time period to provide the desired degree of UV-C disinfection.

In the example of FIG. 6, there are four toothbrush holding chambers 602 shown. In alternate embodiments, the aspects of the disclosed embodiments are not so limited. In alternate embodiments, the apparatus 600 can include any suitable number of toothbrush holding chambers 602 other than including four. For example, larger versions of the apparatus 600 could include more than four chambers 602. Smaller versions, or portable versions of the apparatus 100 could include less than four chambers 602.

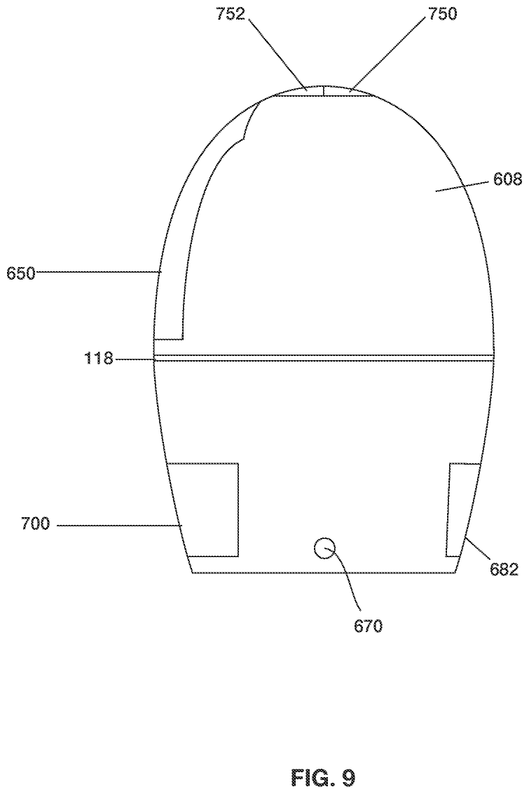

Referring also to FIGS. 6 and 9, in one embodiment, the apparatus 600 includes a tank 614. The tank 614 is in fluid communication with the chamber(s) 602 and the drainage pipe 604. In this example, fluid, water and debris can flow from the chamber(s) 602 and into the tank 614. The tank 614 can be used to collect the fluid, water and debris until it can be drained using the drain pipe 604. In one embodiment, a height of the tank 614 can be approximately ¾ inches. In alternate embodiments, the height and size of the tank 614 can be of any suitable dimensions.

In one embodiment, a chamber 602 will have a fluid or drainage connection to the tank 614. This can be in the form of a drainage hole or opening, or other suitable drainage port. In one embodiment, the bottom wall or floor of a chamber 602 can be sloped or angled. In this manner, the angle will promote the drainage away from the head of the brush and from the chamber 602 to the drainage tank or other drain, such as the drain pipe. The slope can be any suitable slope that promotes the drainage from the chamber 602.

In one embodiment, a chamber 602 will include one or more drain holes (not shown). The drain holes can be disposed on a bottom or sidewall of the chamber 602. The drain holes are configured to allow water and debris from the toothbrush to collect and drain out. The collected water is delivered to the drain pipe or tube 604. In one embodiment, the waste from a chamber 602 is first drained to and collected in the tank 614. From there, the collected water can be drained to a sink, for example, via the drain pipe 604.

The tank 614 will be fluidly coupled to the drainage pipe 604. In one embodiment, a floor or bottom of the tank 614 can be sloped to promote the drainage of any fluid and debris from the tank 614 and out the drainage pipe or other drainage port that can be coupled to the tank 614.

In certain examples, the apparatus 600 can include a drain pipe 604 that will extend and tuck away in a range of 0 degrees to and including approximately 90 degrees to drain fluid and debris collected from the apparatus 600 into a sink or other fluid disposal. For instance, the drain pipe 604 can be fluidly coupled to the waste tank 614 shown in FIG. 6 that is configured to capture or collect waste water from the individual chamber. The drain pipe 604 will be used to drain the tank 614. In one embodiment, the drain pipe 604 can be angled or sloped downwards to assist in any draining. In one embodiment, the drain pipe 604 is a flexible tube that allows the drain pipe 604 to be readily and removable placed in or connected to a sink or other waste disposal facility. In one embodiment, the apparatus 600 can include an access door or panel that is used to gain access to the drain pipe 604. In this manner, the drain pipe 604 can remain out of sight until used.

In certain examples, the chamber 606 can have a different shape to accommodate the shape and size of a head of a razor, as compared to the shape and size of a toothbrush. In one embodiment, the chamber 606 can also include two or more water spray nozzles or jets, as well as a germicidal disinfectant light(s).

In certain examples, the apparatus may support and a tube of toothpaste. In alternate embodiments, the apparatus 600 can configured to hold a plurality of toothbrushes, as well as other teeth cleaning or other products. In the example of FIG. 9, there are chambers for four toothbrushes, or three toothbrushes and a razor. In alternate embodiments, the apparatus 600 can be configured to hold any suitable number and combination of toothbrushes, razor and other products. For example, in one embodiment, the apparatus 600 can be configured to hold four toothbrushes, one razor, a tube of toothpaste and possibly dental floss.

In one embodiment, toothbrush(es) and/or razor can be held in place with a spring loaded pressure plate to keep the toothbrush from moving during cleansing and sanitizing. In one embodiment, the pressure plate assembly can be connect to a sensor that is used to detect when a toothbrush is properly inserted. In alternate embodiments, any suitable sensor, such as an optical sensor, can be used to detect when a toothbrush is properly inserted.

In certain examples, the casing 608 can act as a protective cover. The protective cover will help keep the toothbrush(es) and razor free from dust, dirt, bathroom cleaners over spray, toilet plumage, germs floating in the air and other bodily sprays. Further, in one embodiment, the apparatus 600 can include an access door that will allow the user to access the toothbrush holding chambers. In one embodiment, the pressure spray and germicidal light will not be able to be activated unless the access door is closed. In this example, one or more sensors can be used to detected one or more of the closed and/or open state of the door(s). Closing the access door can complete the circuit and enable pressure washing and light disinfection. In this manner, the access door can be used to protect people from the dangers of being exposed to the high pressure spray and UV-C.

In one embodiment, the activation buttons and indicator lights can be configured with a black tint to camouflage them. The buttons can be back lit for about 15 seconds when the access door is closed so you can see them. In one embodiment, the hand wand will have a button and will be configured to be able to be activated with the access door open.

In one embodiment, the cleaning cycles for the toothbrush(es) can be programmed and automatic. For example, the cleaning cycle can be programmed to automatically begin at a certain time each day. In one embodiment, a cleaning cycle can begin when one or more toothbrushes are disposed in the apparatus for a pre-determined period of time. In one embodiment, a cleaning cycle can be programmed to begin after it is detected that a toothbrush has been removed and inserted a pre-determined number of times. Any suitable criteria can be used to determine when to start a cleaning cycle. In these examples, the apparatus 600 will include a suitable controller(s) with processor(s) and memory, that is configured to carry out one or more of the method steps described herein, either alone or in combination.

In other embodiments, the disclosure includes a sanitation station kit. In such an embodiment, the kit may comprise a sanitizing assembly, e.g. any of the sanitizing assembly elements and components previously shown or described; a germicidal UV light illumination, e.g. any of the germicidal UV light illumination elements and components previously shown or described. Further, other embodiments of the kit may comprise a housing, e.g. any combination of housing elements and components previously shown or described.

Numerous characteristics and advantages have been set forth in the foregoing description, together with details of structure and function. Many of the novel features are pointed out in the appended claims. The disclosure, however, is illustrative only, and changes may be made in detail, especially in matters of shape, size and arrangement of parts, within the principle of the disclosure, to the full extent indicated by the broad general meaning of the terms in which the general claims are expressed. It is further noted that, as used in this application, the singular forms “a,” “an,” and “the” include plural referents unless expressly and unequivocally limited to one referent.

What is claimed is:

1. A sanitation station, said station comprising:
a. a housing having at least one access door;
b. a sanitizing assembly housed within said housing and adapted to sanitize at least one personal hygiene product being accessible about said access door; and
c. an activation assembly integrated about said housing and adapted to activate said sanitizing assembly.

2. The assembly of claim 1, wherein said housing includes an upper dual switch gate, wherein said upper dual switch gate comprises:
a. a first portion release mechanism adapted to release said access door; and
b. a second portion aperture adapted to expose a solution fill inlet.

3. The assembly of claim 1, wherein said sanitation station adapted to provide a sanitization to ensure said personal hygiene product is at least one of disinfected, sanitized, and sterilized according to a corresponding output indication.

4. The assembly of claim 1, wherein said housing comprises an oval shape.

5. The assembly of claim 4, wherein said access door having a concave elongated surface extending along an upper portion of said oval housing.

6. The assembly of claim 1, wherein said housing comprises a non-oval shape.

7. The assembly of claim 1, wherein said housing comprises a night light.

8. The assembly of claim 7, including a motion sensor in electrical communication with said night light.

9. The assembly of claim 7, including a plurality of color illuminations.

10. The assembly of claim 7, including a plurality of adjustable brightness levels.

11. The assembly of claim 1, wherein said housing comprises a slide cover.

12. The assembly of claim 11, wherein said slide cover includes a spring-loaded slide cover adapted to release in an opening position and apply pressure in a closed position.

13. The assembly of claim 1, wherein said housing comprises a discharge tube.

14. The assembly of claim 13, wherein said discharge tube includes a plurality of venting holes adapted to evaporate moisture.

15. The assembly of claim 1, wherein said activation assembly comprises a user control interface.

16. The assembly of claim 15, wherein said user control interface includes a control adapted to activate a heated, pressurized solution washing assembly.

17. The assembly of claim 16, wherein said user control interface adapted to activate said heated, pressurized solution washing assembly in an individual chamber.

18. The assembly of claim 15, wherein said user control interface includes a control adapted to activate a germicidal light illumination.

19. The assembly of claim 18, wherein said user control interface adapted to activate germicidal light illumination in an individual chamber.

20. The assembly of claim 1, wherein said housing comprises a low solution tank indicator.

21. The assembly of claim 1, wherein said housing comprises an open door indicator.

22. The assembly of claim 1, wherein said housing comprises at least one external support slot.

23. The assembly of claim 22, including a toothpaste slot.

24. The assembly of claim 1, wherein said housing comprises an audio speaker adapted to provide at least one sound effect.

25. The assembly of claim 1, wherein said sanitizing assembly adapted to provide a pressurized fluid sanitization to ensure said personal hygiene product is at least one of disinfected, sanitized, and sterilized according to a corresponding output indication.

26. The assembly of claim 1, wherein said sanitizing assembly includes a fluid sanitizing system.

27. The assembly of claim 26, wherein said sanitizing assembly includes at least one pressurized output port adapted to direct a pressurized stream of solution at said personal hygiene product.

28. The assembly of claim 1, wherein said sanitizing assembly includes a germicidal light illumination adapted to direct a sanitizing illumination at said personal hygiene product.

29. The assembly of claim 28, wherein said germicidal light illumination includes at least one ultraviolet (UV) light source adapted to project a UV light for at least one of disinfection, sanitization, and sterilization of said personal hygiene product.

30. The assembly of claim 1, wherein said sanitation station being an independent, portable device adapted to align on a bathroom surface.

31. A hygiene device comprising:
a. a plurality of support compartments adapted to receive a personal hygiene product;
b. a heated, pressurized solution washing assembly in fluid communication with said support compartments; and
c. a germicidal light illumination in optical communication with said support compartments.

32. The device of claim 31, including a solutionpik fluidly coupled to said heated, pressurized solution washing assembly and adapted to deliver a solution stream.

33. The device of claim 32, wherein said solutionpik includes a tube in fluid communication with a solution reservoir.

34. The device of claim 31, wherein said plurality of support compartments comprise at least one toothbrush aperture adapted to temporarily secure a neck of a toothbrush in an operating position.

35. The device of claim 34, wherein said toothbrush aperture comprising a knob and spring-loaded system adapted to provide a pressure against said neck of said toothbrush.

36. The device of claim 34, including a plurality of toothbrush apertures.

37. The device of claim 36, wherein said plurality of toothbrush apertures being spatially aligned adjacent one another.

38. The device of claim 31, wherein said plurality of support compartments comprise at least one razor aperture.

39. The device of claim 38, including a plurality of razor apertures.

40. The device of claim 31, wherein said heated, pressurized solution washing assembly adapted to provide a pressurized fluid spray of at least one of about one hundred pounds per square inch distributed about a plurality of spray nozzles; about one hundred pounds per square inch about an individual spray nozzle; about fifty pounds per square inch about an individual spray nozzle; greater than about one hundred pounds per square inch about an individual spray nozzle; and a combination thereof.

41. The device of claim 40, including a switch to toggle between a desired solution pressure.

42. The device of claim 31, wherein said heated, pressurized solution washing assembly comprises a tank configured to store washing solution supplied to said support compartments.

43. The device of claim 31, wherein said heated, pressurized solution washing assembly includes a pump adapted to supply a washing solution stored in the tank to a spray nozzle.

44. The device of claim 31, wherein said heated, pressurized solution washing assembly includes at least one pressurized output port adapted to direct a pressurized fan of solution at said personal hygiene product.

45. The device of claim 31, wherein said heated, pressurized solution washing assembly includes a solution heating device coupled to a solution pump and adapted to heat said solution to a desired effluent temperature.

46. The device of claim 45, wherein said desired effluent temperature includes about one hundred and forty degrees Fahrenheit.

47. The device of claim 44, wherein said solution comprises water.

48. The device of claim 31, wherein said heated, pressurized solution washing assembly includes a plurality of spray nozzles.

49. The device of claim 31, wherein said heated, pressurized solution washing assembly includes a port in fluid communication with a manifold adapted to selectively direct solution to associated nozzles.

50. The device of claim 31, wherein said heated, pressurized solution washing assembly includes a nozzle adapted to project a pressurized steam.

51. The device of claim 50, wherein said pressurized steam adapted to wash at least one chosen from a debris on a toothbrush and a debris on a razor.

52. The device of claim 49, including a Y-split tubing having respective distal nozzles.

53. The device of claim 31, wherein said germicidal light illumination adapted to direct a sanitizing illumination at said support compartments.

54. The device of claim 31, wherein said germicidal light illumination includes at least one ultraviolet (UV) light source adapted to project a UV light for at least one of disinfection, sanitization, and sterilization of said personal hygiene product.

55. The device of claim 54, wherein said germicidal light illumination adapted to provide a dosage of UV light emitted by the UV light source to ensure said personal hygiene product is at least one of disinfected, sanitized, and sterilized.

56. The device of claim 54, wherein said UV light source is adapted to emit UV light having a wavelength from about 50 nanometers to about 500 nanometers.

57. The device of claim 54, wherein said germicidal light illumination comprises a UV light source of at least one of a UV light emitting diode (LED), UV LED array, fluorescent bulb, a laser, and a combination thereof.

58. The device of claim 31, including a housing.

59. The device of claim 58, wherein said housing comprises an oval shape.

60. The device of claim 31, including an activation assembly.

61. The device of claim 31, including a funnel adapted to distribute a solution to said heated, pressurized solution washing assembly.

62. The device of claim 31, wherein said hygiene device being an independent, portable device.

63. The device of claim 31, wherein said plurality of support compartments being independent.

64. The device of claim 63, wherein said independent support compartments comprise at least one toothbrush support compartment being independent of a razor support compartment, and whereby said independent support compartments adapted to eliminate cross contamination of said toothbrush support compartment about said razor support compartment.

65. A device comprising:
a. an oval housing having at least one concave access door and supporting a plurality of internal compartments adapted to receive a plurality of personal hygiene products;
b. an adjustable sanitizing assembly positioned within said housing, wherein said adjustable sanitizing assembly comprising:
i. a heated, pressurized solution washing assembly in fluid communication with said internal compartments; and
ii. a germicidal UV light illumination in optical communication with said support compartments; and
c. a removable solutionpik adapted to deliver a solution stream, and
wherein said sanitizing assembly adapted to sanitize at least one personal hygiene product being accessible about said concave access door.
